(19) United States
(12) Patent Application Publication
(10) Pub. No.: US 2011/0136147 A1
(43) Pub. Date: Jun. 9, 2011

(54) Methods of identifying tumor associated glycoproteins

(76) Inventor: J. Michael Pierce, Athens, GA (US)

(21) Appl. No.: 11/661,743

(22) PCT Filed: Sep. 7, 2005
(86) PCT No.: PCT/US05/31650

§ 371 Date: Feb. 29, 2008

Related U.S. Application Data
(60) Provisional application No. 60/607,952, filed on Sep. 8, 2004.

(51) Int. Cl. G01N 33/574; C07K 14/82

(52) U.S. Cl. 435/7.23; 530/395

(57) ABSTRACT

Novel methods to identify and characterize glycoproteins that are likely to be found in serum, and that will therefore provide more relevant disease markers than current methods are disclosed. The proteins that are likely to be found in serum are distinguished either by cleavage from the membrane or as potentially secreted proteins characterized by solubility in an aqueous phase. The methods disclosed also allow determination of differential glycosylation patterns of proteins that are likely to be found in serum from cells in various stages of disease. In addition, methods to identify novel therapeutic targets by identifying glycoproteins retained in diseased cells are disclosed.

### RELATION TO PRIOR APPLICATIONS

The present application claims priority to U.S. Ser. No. 60/607,952 filed Sep. 8, 2005, the contents of which are hereby incorporated by reference.

### FIELD OF THE INVENTION

The present invention relates to methods for identifying glycoproteins associated with disease states such as cancer, in which the glycoproteins are characterized by an altered glycosylation pattern.

### BACKGROUND OF THE INVENTION

Recent statistics indicate that cancer kills approximately 550,000 individuals per year in the US alone. While mortality from different cancers varies widely, the major factor influencing mortality rates is time to diagnosis and identification of metastases. There is a strong need for well defined and easily administered diagnostic tests that can identify cancers in their early stages. Current diagnostic tests are often inaccurate and invasive. In the case of the commonly used cancer marker prostate specific antigen, for example, levels of normal expression of this protein vary dramatically between individuals, and current tests are only beginning to address this issue (See U.S. Pat. No. 6,261,791, issued Jul. 17, 2001).

### 1. Serum Availability

Currently, only a small number of cancer-specific markers that are present in the serum of patients are known. A useful tumor marker is present either in considerably greater quantity in tumor cells or is expressed exclusively in tumor cells, and is measurable in the serum from patients. However, in identifying potential diagnostic markers, investigators to date have focused almost entirely on proteins that are expressed in tumor cells without focusing on the potential serum availability of these markers. There are two principal modes of release of cells into the serum: cleavage from the extracellular leaflet of the plasma membrane and release of soluble proteins from secretory vesicles.

The release from the extracellular leaf of the plasma membrane can occur through cleavage of a glycosylphosphotidylinositol (GPI) linkage. This linkage is achieved through a phosphatidylinositol anchor, which binds a variety of proteins to the external leaflet of the plasma membrane via a glycosyl bridge. In this case the protein is attached to the carbohydrate through phosphatidylethanolamine (PE) linkage, and the carbohydrate is in turn attached to the membrane via linkage to phosphatidylinositol (PI), which anchors the structure within the membrane. The linkage is called a GPI “anchor” and is common to a variety of proteins on the extracellular surface. Proteins which are destined to be linked to the membrane via a GPI anchor contain a recognition sequence comprising approximately 20 mainly hydrophobic amino acids at the carboxyl terminus of the protein, much of which is cleaved during GPI linkage (see Eisenhaber, et al., 1998; U.S. 20020058288A1 and references therein). Because these proteins are linked via PI to the membrane, they are readily cleaved by phospholipases, including phospholipases C and D, leaving their aliphatic residues embedded in the membrane. These proteins are therefore hydrophobic only until their GPI anchor is cleaved, releasing the hydrophilic protein into the extracellular matrix. Proteins that are GPI-anchored include hydrolytic enzymes, cell surface antigens, and proteins involved in interactions with other cells. In U.S. Pat. No. 5,268,272, Mullner et al disclose a method for making GPI-linked proteins water soluble by cleaving the inositol linkage with bacterial phospholipases, while, Jeffries et al. (U.S. Pat. No. 6,455,494) discloses isolating a cleaved form of the GPI-anchored p97 protein by incubating cells expressing GPI-anchored p97 with an enzyme that cleaves GPI anchors and isolating the cleaved form. Neither of these references disclose the identification or purification of disease markers, or the isolation of lectin-binding glycoproteins.

Studies of GPI anchored proteins have shown that these proteins are cleaved into media by prokaryotes, and a recent study also identified the cleavage of an extracellular GPI-linkage in humans (Park S W, et al. 2002). While this is the first direct evidence for this cleavage in humans, glycosylphosphatidylinositol-specific phospholipase D (GPI-PLD) is abundant in serum and is known to cleave GPI-anchored proteins from cell membranes (Deeg and Davitz, 1995). Endogenous PLD may also be and part of the mechanism for protein turnover in cells.

It is also possible to identify secreted proteins from a sample of bodily fluids based upon the presence of a secretion signal sequence in the RNA that encodes an isolated protein. As is known in the art of cellular biology, certain proteins are expressed and destined for secretion through secretory vesicles. A secretion sequence is typically present on these proteins when they are initially expressed, but cleaved from the protein before the protein is secreted. By matching an isolated to its corresponding RNA, one can determine whether the protein is a secreted protein based upon whether the RNA encodes a secretion sequence.

### 2. Glycoprotein Identification

The covalent attachment of oligosaccharides to protein is the most common post-translational event and occurs in more than 50% of proteins, independent of membrane linkage. Glycosylation occurs at specific locations along the polypeptide backbone of proteins. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be any amino acid except proline. N-acetylneuraminic acid (also known as sialic acid) is usually the terminal residue of both N-linked and O-linked oligosaccharides. Variables such as protein structure and cell type influence the number and nature of the carbohydrate units within the chains at different glycosylation sites. Structural variations in oligosaccharides are also common at the same site within a given cell type. The types and levels of glycosylation are a reflection of the levels and activities of different glycosyltransferases and glycosidases responsible for the intracellular construction of oligosaccharides. Numerous secretory proteins, including immunoglobulins, are glycoproteins, as are most components of plasma membranes such as cell membrane receptors, where the carbohydrates can be involved in cell-to-cell adhesion directly or can regulate such adhesion. There are several techniques available to identify glycosylated peptides from cell lysates. The principal techniques rely on the binding of glycan chains to specific lectins. One method that has been extensively employed is serial lectin-affinity chromatography to purify glycoproteins from cell extracts (see for examples Endo, 1996; Jaques et al., 1996; Yoshida et al., 1997; Sumi et al., 1999; Yoshida et al., 1999; Hoja-Lukowicz et al., 2001). In these studies, extracts of tissue were bound to several different lectin-agarose columns, and subsequently eluted off these columns by haptene competition. Recovered proteins can be subjected to proteolysis and re-absorbed to lectin columns to increase specific binding. Purified proteins can subsequently be identified by proteolytic cleavage and mass spectrometry. Although these studies show purification and identification of lectin-bound glycoproteins, they fail to disclose methods which would allow separation of lipid-bound from non-lipid bound proteins. These methods are also not designed to identify tumor-specific proteins that could be used as disease markers.

### 3. Glycoproteins as Disease Markers

There is now overwhelming evidence that glycosylation of the glycoproteins produced by tumor cells is markedly altered (see for example Dwek et al., 2001; Hanisch, 2001; Hakomori, 2002). Altered glycosylation is a common feature in the transformation to malignancy of certain cancers and has been related to the invasiveness and metastatic potential of tumor cell lines. Investigators have applied this fact to look for cancer specific carbohydrate markers. In most of these studies, cell extracts were separated by SDS-PAGE and probed using a lectin conjugated for visualization. The levels of binding of the lectin could then be compared between tumor and control cells to identify protein bands with increased levels of glycosylation that could be excised for subsequent sequencing. These studies fail to focus on proteins that are likely to be released by tumor cells. Because the goal of diagnostic tests is early detection and the ability to screen large numbers of patients, serum availability of diagnostic markers is an important criterion.

Previous studies have shown that changes in glycosylation have the potential to be used as diagnostic tools for cancers and other disease states. Some studies have even attempted to identify glycoproteins having altered glycosylation patterns in various disease states. However, these studies failed to distinguish between glycoproteins that are simply present in tissue and glycoproteins that have a high likelihood of being present in the serum. There is therefore a need for methods that limit the range of potential disease markers to those that are likely to be available in patient serum and therefore most likely to provide valuable diagnostic tools. The need extends to selecting the most promising markers of disease, which are likely to be glycoproteins.

### OBJECTS OF THE INVENTION

Accordingly, it is an object of the present invention to provide a method for identifying novel glycoprotein markers that have a high probability of being released from diseased tissue or cells, particularly diseased tumor cells, into serum.

It is another object of the present invention to narrow the pool of proteins present a tissue sample to a subset of glycoproteins that have a high probability of being released into serum.

Another object of the present invention is to identify glycoproteins from a cell or tissue sample that are present on the cell membrane and that have a high probability of being released into serum and can act as disease markers.

Yet another object of the present invention is to identify glycoproteins from a cell or tissue sample present within secretory vesicles inside the cell that have a high probability of being released into serum and can act as disease markers.

Another object of the present invention is to provide methods for identifying glycoproteins that can act as disease markers because they possess glycosylation patterns that differ from the glycoproteins released from non-diseased cells.

A further object of the present invention is to provide methods for identifying proteins that are not secreted or released from cells into serum to identify potential therapeutic targets of disease.

A further object of the present invention is to provide methods to identify tissue-bound glycoproteins that can be used to distinguish diseased tissue specimens.

Yet another object of the present invention is to provide methods for identifying glycoproteins that can act as disease markers because they are differentially expressed in diseased versus non-diseased cells.

### SUMMARY OF THE INVENTION

These and other objects are achieved by methods to selectively separate, from a cell or tissue sample, those glycoproteins that are most likely to be associated with disease states. These methods narrow the pool of proteins present in a sample of tissue to two subsets of glycoproteins: 1) those that are likely to be found in serum; and can thus function as disease markers in readily administered diagnostic tests; and 2) those that are likely to remain in the cell and can thus function as diagnostic markers in excised tissue samples or can be used to target therapeutic compounds to diseased tissue.

In one embodiment the invention provides a method for identifying cell-membrane glycoproteins that are released into serum. The method takes advantage of the fact that glycoproteins that have GPI anchors are likely to be released into serum when the GPI anchor is cleaved by phospholipases in vivo. The method is performed in vitro by (a) providing a plurality of cells harboring a disease; (b) incubating said cells with an enzyme that cleaves glycolipid anchors such as a phospholipase to yield cleaved glycoproteins that comprise an inositol moiety and uncleaved glycoproteins; and (c) separating said cleaved glycoproteins from said uncleaved glycoproteins. The enzyme used in the method is preferably capable of cleaving a GPI-anchor, and preferably is PI-PLC. The cleavage of the GPI anchor from the glycoprotein exposes an inositol residue on the glycoprotein that can be used as a target to separate the cleaved glycoproteins from the non-cleaved glycoproteins through conventional techniques such as immunoprecipitation using an antibody reactive to the inositol moiety.

In another embodiment, the invention provides a method for separating glycoproteins in the inside of a cell that are likely to be secreted into serum from those that will remain in the cell. This method relies upon the solubility differences between the hydrophilic contents in the interior of the cell and the lipophilic contents of the cell membrane to narrow the set of potential glycoprotein markers in a cell sample to glycoproteins that are inside the cell. Thus, in this embodiment the invention provides a method for isolating serum glycoprotein markers that are associated with a disease state comprising: (a) providing a plurality of cells harboring a disease; (b) solubilizing said cells in a liquid medium comprising water and a detergent; (c) separating said liquid medium into an aqueous phase comprising hydrophilic proteins and a detergent phase comprising lipophilic proteins; and (d) separating glycoproteins from non-glycoproteins in said aqueous phase. This phase separation method is based on the ability of the detergent to partition into two phases: a detergent rich phase and a detergent poor phase due to temperature changes. When a detergent such as Triton X-114 with a defined cloud point is employed, the soluble glycoproteins from the interior of the cell are present exclusively in the aqueous phase after phase separation. Once the proteins from the interior of the cell are isolated, the pool of candidate markers can further be narrowed by separating glycoproteins from non-glycoproteins using lectin affinity columns, and can still further be narrowed by identifying glycoproteins from the pool that comprise secretory amino acid sequences. Those proteins that remain in the detergent fraction have a low probability of release and can therefore be analyzed to function as tissue-bound disease markers.

Once a subset of glycoproteins, either those likely to be released from the cell or those likely to remain bound to the cell membrane, is isolated from the larger pool of cellular proteins, this subset can be further analyzed to assess their ability to act as disease markers. Methods for assessing the ability of these glycoproteins to serve as tumor markers include analyzing the glycosylation profiles of the isolated glycoproteins for aberrant glycosylation patterns. This analysis may include comparing the glycosylation profiles of glycoproteins isolated from diseased cells with the glycosylation profiles of glycoproteins isolated from non-diseased cells. The ability of an isolated glycoprotein to act as disease marker can also be determined based upon the level of expression of any particular glycoprotein, and/or the pattern of expression of multiple glycoproteins. Once again, these analyses may include comparing the expression levels and/or patterns of glycoproteins isolated from diseased cells with the expression levels and/or patterns of glycoproteins isolated from non-diseased cells.

In addition, this invention provides a tool to identify potential therapeutic markers for disease by analysis of those glycoproteins that are not released from the cell, therefore representing proteins that can be found differentially expressed or glycosylated in diseased cells and can be targeted with, for example, cytotoxic agents.

Thus, the present invention allows the identification of diagnostic markers that will provide a basis for the development of non-invasive diagnostic tests. These methods are more fully described and exemplified below.

### DETAILED DESCRIPTION OF THE INVENTION

One embodiment of the present invention provides a method for isolating membrane-bound glycoproteins that are likely to be released into serum comprising (a) providing a plurality of cells harboring a disease; (b) incubating said cells with a phospholipase to yield cleaved glycoproteins that comprise an inositol moiety and uncleaved glycoproteins; and (c) separating said cleaved glycoproteins from said uncleaved glycoproteins.

### 1. Phospholipase-Cleavage

The cleavage step is specific for glycoproteins that comprise a GPI anchor, and is intended to yield glycoproteins on which an inositol residue from the GPI anchor is exposed. Any of the enzymes that display specificity toward GPI linkages may be utilized within the context of the present invention to cleave the GPI anchor. The enzymes that cleave and release glycoproteins or carbohydrates that are membrane-anchored via linkage to phosphatidylinositol include, but are not limited to, phospholipases of type C and D. Representative examples include phosphatidylinositol specific phospholipase C, also known by the abbreviation PI-PLC or as 1-phosphatidylinositol phosphodiesterase, GPI-PLCs, and glycosyl-phosphatidylinositol-specific phospholipase D, or GPI-PLD (see generally, Ferguson and Williams, 1988). The GPI-PLD and PI-PLC enzymes have been described from eukaryotic sources (See Low, 1990; Low and Prasad, 1988; Essen et al., 1996; and Essen et al., 1997).

For this embodiment of the invention, one of two conditions will typically be observed: (1) the conditions of the first buffer will vary depending on the requirement of the specific phospholipase, or (2) the first buffer will be diluted to be compatible with enzymatic cleavage. For example, in some cases GPI-PLD requires calcium and zinc, but not Mg⁺² ions in the dilution buffer to stabilize the enzyme activity (Li et al., 1994).

In a preferred embodiment, the enzyme that cleaves phospholipids anchors is phospholipase C (PI-PLC). PI-PLC has been described from prokaryotic sources, including extracellular production by bacteria. Among the known bacterial sources of PI-PLC are Bacillus cereus (Slein and Logan, 1967; Hetland and Prydz; 1982; Ikezawa et al. 1976; Volwerk et al., 1989; and Kuppe et al., 1989), Bacillus thuringiensis (Japanese patent document JP 55034039), Staphylococcus aureus (Low and Finean, 1977), and Clostridium novyi (Taguchi and Ikezawa, 1978) or from recombinant sources (Koke et al., 1991; and Henner et al., 1988).

### 2. Inositol Separation

The separation of cleaved glycoproteins from the uncleaved glycoproteins preferably occurs by virtue of this free inositol moiety. The inositol 1,2-cyclic monophosphate moiety released by phospholipase cleavage of a GPI anchor has been referred to as the cross-reacting determinant (CRD), and can be readily detected by antibodies that are either commercially available or independently developed by techniques well known in the art. In addition to CRD-specific antibodies, other affinity recognition tools such as aptamer recognition or ion exchange chromatography can be utilized to separate cleaved glycoproteins, without affecting the essence of the invention.

In one embodiment, the cleaved glycoproteins are separated from non-cleaved glycoproteins by immunoaffinity using an antibody to the inositol moiety using standard immunoprecipitation techniques well known in the art. In a preferred embodiment the method comprises (a) contacting the cleaved and uncleaved glycoproteins with a first antibody that recognizes the inositol moiety to form antibody-glycoprotein complexes; (b) incubating the antibody-glycoprotein complexes with a capture molecule such as protein G that recognizes the first antibody and that preferably is attached to a stable support such as agarose or sepharose beads; (c) optionally centrifuging the resulting complex briefly and washing the complex with a wash buffer; and (d) eluting the cleaved glycoproteins that have been captured with an elution buffer.

The elution buffer preferably comprises a denaturing agent. Exemplary denaturing agents include agents that disrupt disulfide bonds such as dithiothreitol (DTT) and ionic detergents such as sodium dodecylsulfate (SDS). Alternatively, the elution buffer can be prepared at a pH that will denature proteins, or it may contain a chaotropic agent that will disrupt the contact between the inositol moiety and the antibody. In another embodiment, the elution buffer comprises a concentration of free inositol in excess of the concentration of cleaved glycoprotein.

In a separate embodiment, cleaved glycoproteins are separated from non-cleaved glycoproteins by immunoprecipitation comprising contacting the cleaved and uncleaved glycoproteins with a first antibody that recognizes the inositol moiety in which the antibody itself is covalently attached to a stable support such as sepharose beads. The resulting, glycoprotein/antibody complex is washed with a standard buffer, and cleaved glycoproteins are eluted using an elution buffer as described above.

2. Pretreatment of Cells with a Buffer

In one embodiment of the invention, the diseased cells are incubated in a first buffer before incubation with a phospholipase in order to disrupt the cellular and subcellular structures, preferably via solubilization. Conditions in the first buffer which can be altered include, but are not limited to: the ionic strength; the detergent concentration; the concentration of chaotropic agents, and the pH. In one embodiment of the invention, the ionic strength of the first buffer is varied. Higher ionic strength buffers will decrease the interactions of glycoproteins with polyamines and any electrostatic interaction between glycoproteins and other proteins. Suitable ranges in which to vary the destabilizing agent concentrations can readily be determined. For example, in order to alter ionic strength, potassium chloride can be titrated from about 0.05M to 2M. The range of pH can be titrated from about 2 to 10.

In another embodiment of this invention, the cells are pretreated in a buffer that comprises a chaotropic agent for the removal of non-membrane bound proteins. This technique preferably yields a mixture of glycoproteins that is more highly concentrated in membrane-bound glycoproteins. Chaotropic agents include, but are not limited to, urea, urea, guanidine, guanidinium salts such as Guanidinium Thiocyanate (GuSCN) and Guanidinium Hydrochloride (GuHCl), sodium iodide, and potassium thiocyanate. Chaotropic agents can be titrated from about 0.5M to 8M.

### 3. Cell Solubilization

In a further embodiment of this invention, the first buffer contains at least one detergent for solubilizing at least a portion of the membrane protein components. A membrane protein is considered solubilized if it is present in the supernatant after one hour centrifugation of a lysate or a homogenate at 100,000×g. For as yet unknown reasons, specific detergents often work better for particular isolation procedures. For example, EMPIGEN BB® (Calbiochem) has been found to be the most efficient detergent in solubilization of keratins while preserving their antigenicity. Similarly, n-Dodecyl-β-D-maltoside (Calbiochem) has been found to be the detergent of choice for the isolation of cytochrome c oxidase. Hence, some “trial and error” may be required for determining the most optimum conditions for solubilizing different glycoproteins found in cells. In some cases, it has been observed that the inclusion of nondetergent sulfobetaines with detergents in the isolation buffer improves yields of solubilized membrane proteins.

Detergents used for solubilization can include any of a variety of ionic and non-ionic detergents. Detergents, in particular, detergents originally designed and tested for their ability to solubilize biomolecules may be used. Examples of detergent classes and detergents that can be used for solubilization include, but are not limited to anionic detergents (such as Linear alkylbenzene sulfonate, Alkyl sulfates, alpha.-Olefin sulfonates, Alcohol ether sulfates, Sulfosuccinates, Phosphate esters, Fatty acid salts, Perfluorocarboxylic acid salts, Abietic acid), cationic detergents (such as Cetyl trimethylammonium bromide, Alkylated pyridium salts), zwitterionic detergents (such as alkyl betaine, CHAPS), neutral detergents (such as Alkyl phenol PEG Alkyl PEG, Alkanolamides, Glycol and Glycerol esters, Propylene glycol esters, Sorbitan and PEG sorbitan esters, Polydimethylsiloxan PEG), amphoteric detergents (such as Dodecyl dimethyl amine oxide), and polymeric detergents (such as polyacrylic acid).

In one embodiment, the cells are incubated in a non-ionic detergent such as Triton X-100 or Brij96 under cold conditions (as described in Madore et al, 1999) to solubilize membrane components other than those found in detergent-insoluble glycosphingolipid/cholesterol-enriched microdomains (DIGs), which are known in the art to be enriched in GPI-anchored proteins and for which there is reason to believe that these may be dynamically regulated, particularly in disease. In this embodiment the solubilized cells are separated by centrifugation into detergent-extractable and detergent-insensitive components. Each component can then be individually analyzed for phospholipase-sensitive glycoproteins. Detergents, either ionic or non-ionic, can be titrated from about 0.05% to 2%.

After solubilization of the cells, one embodiment of the invention allows recognition of GPI-anchored proteins by virtue of affinity to their recognition sequence. Because GPI-anchoring sequences vary for different classes of molecules, affinity reagents including antibodies can be used to affinity purify these proteins. However, many of these sequences are known in the art and specific reagents can be designed to individual sequences, or to the upstream amino acid triplet termed “omega to omega-2” which designates the site of GPI uptake.

### 4. Secreted Glycoproteins

A further embodiment of the invention provides a method to isolate water-soluble glycoproteins that are not anchored to the cell membrane. Soluble glycoproteins are hydrophilic and therefore are present exclusively in the aqueous phase after detergent phase separation. In this embodiment, cell extracts are prepared by a method comprising: (a) providing a plurality of cells harboring a disease; (b) solubilizing said cells in a liquid medium comprising water and a detergent; (c) separating said liquid medium into an aqueous phase comprising hydrophilic proteins and a detergent phase comprising lipophilic proteins; and (d) separating glycoproteins from non-glycoproteins in said aqueous phase.

In this embodiment, cells are solubilized with a detergent that exhibits a cloud point, preferably Triton X-114. At a particular temperature above the critical micellar temperature, non-ionic detergents become cloudy and undergo phase separation to yield a detergent-rich layer and an aqueous layer. This temperature is called the “cloud point”. Phase separation presumably occurs due to a decrease in hydration of the head group. For example, the cloud point of Triton X-100 is 64° C. whereas that for Triton X-114 is around 22° C. (for a 1% solution in water, it is 20-22° C.). Hence, Triton X-114 solutions are maintained cold. This property can be used to a particular advantage. Membranes can be solubilized at 0° C. and the solution can be warmed to about 30° C. to effect the phase separation. This allows partition of integral membrane proteins into the detergent rich phase which can be later separated by centrifugation. X-114 is a nonionic detergent, which is often used in biochemical applications to solubilize proteins. The “X” series of Triton detergents are produced from octylphenol polymerized with ethylene oxide. The number relates indirectly to the number of ethylene oxide units in the structure. X-114 has 7 to 8 ethylene oxide units per molecule, with an average molecular weight of 537. In addition, lower and higher mole adducts will be present in lesser amounts, varying slightly within supplier's standard manufacturing conditions. (A by-product formed during the reaction is polyethylene glycol, a homopolymer of ethylene oxide. Acid is also added to the product to neutralize the product after the base catalyzed reaction is completed). These detergents are commercially available or may be prepared for this purpose.

In one embodiment of this invention, cells are incubated in a liquid medium comprising a buffered aqueous solution with a non-ionic detergent at a temperature below the cloud point of that detergent and permissive for solubilization. In a preferred embodiment, the detergent is Triton X-114 and the incubation temperature is below about 20° C. Solubilized cells are subsequently separated into an aqueous phase and a detergent phase by heating to an appropriate temperature above the critical micellar temperature. In the preferred embodiment, using Triton X-114, one permissive temperature for phase separation is about 37° C. In the preferred embodiment, the phase separated solubilized cells are centrifuged to separate the aqueous (upper) and detergent (lower) phases.

In a general example, cells are incubated in TS buffer (10 mM Tris-HCl (pH 7.6) and 150 mM NaCl) containing 1% (v/v) Triton X-114 at 0° C. These samples are overlaid on to a chilled sucrose cushion (TS buffer containing 6% (w/v) sucrose and 0.06% Triton X-114) and incubated at 37° C. until the solution became turbid, and subsequently centrifuged at room temperature to separate the aqueous (upper) and detergent (lower) phases. The upper aqueous phase can be removed to another tube and either analyzed or supplemented with more Triton X-114 to 0.5%. This can then be overlaid on to the same sucrose cushion and phase separation carried out again. In this embodiment of the invention, the aqueous phase of the Triton X-114 extraction is subjected to a method to separate glycoproteins from non-glycoproteins, as described herein.

A further embodiment of this invention comprises a) providing a plurality of cells harboring a disease; (b) solubilizing said cells in a liquid medium comprising water and a detergent; (c) separating said liquid medium into an aqueous phase comprising hydrophilic proteins and a detergent phase comprising lipophilic proteins; and (d) separating glycoproteins from non-glycoproteins in said detergent phase. This embodiment of the invention can be used to identify target molecules for therapeutic purposes, since therapeutically useful targets, unlike diagnostic targets, are more likely to be found in the detergent phase rather than the aqueous phase. In the preferred embodiment the detergent has a. cloud point and in the most preferred embodiment the detergent is Triton X-114. GPI-anchored proteins are known in the art to partition to the detergent phase.

A further embodiment comprises: e) contacting the detergent phase with a phospholipase to yield cleaved glycoproteins and uncleaved glycoproteins; and f) analyzing said cleaved and uncleaved glycoproteins. This embodiment narrows the range of proteins that are analyzed in two ways. First, separating the detergent phase, cleaved glycoproteins reduces the non-specific background that may occur without further purification steps. Second, the detergent phase, non-cleaved glycoproteins represent those glycoproteins with the lowest probability of being found in serum, because both hydrophilic and GPI-anchored proteins that are likely to be found in serum are removed before analysis. The uncleaved, detergent phase glycoproteins can therefore be analyzed by any of the methods available to analyze either their glycan moieties or their peptide sequence to yield tissue-bound disease markers or potential therapeutic targets. Once analyzed, useful therapies such as cytotoxic agents can be targeted to these glycoproteins which will only be found in cell membranes.

### 5. Glycoprotein Separation

In a further embodiment of this invention, glycoproteins are separated from non-glycoproteins to further narrow the pool of candidate serum markers. It will be appreciated that the separation of glycoproteins from non-glycoproteins can occur either before or after the cleavage of the GPI anchor and purification of the inositol moiety, or before or after solubilization with most detergents, including Triton X-114. Furthermore, the separation of glycoproteins from non-glycoproteins can be accomplished from either an aqueous or detergent fraction from a phase separation of solubilized cells. In one embodiment, the cells which have been solubilized with detergent are subjected to a purification step in which glycoproteins are separated from non-glycoproteins. Depending on the detergent, the first buffer may be diluted to be compatible with the glycoprotein separation steps. In another embodiment, the glycoproteins that have been cleaved and separated based on the availability of their inositol moiety are separated from non-glycoproteins that were also isolated based on a free inositol moiety.

In one embodiment, said glycoproteins are separated from said non-glycoproteins by a process comprising: (a) contacting said glycoproteins with a lectin bound to a stable support; and (b) eluting bound glycoprotein off said lectin. In this instance glycoproteins are identified as proteins with an attached oligosaccharide of any size, while non-glycoproteins are identified as all other proteins in the cell extract. A preferred method for achieving the glycoprotein purification is affinity chromatography e.g. Fast Performance Liquid Chromatography (FPLC), by virtue of the affinity of their carbohydrate moieties to different lectins that can be conjugated to immobilized beads. In this application, a lectin is defined as a sugar-binding protein of non-immune origin that precipitates glycoconjugates and contains at least one sugar-binding site. In addition to conjugated lectins, other non-lectin sugar binding proteins including sugar-specific enzymes and transport proteins can be immobilized and used in this method.

When the cells are solubilized, the buffer used in the solubilization step should be chosen based on standard methodologies adequate for lectin binding chromatography. If the first buffer is not compatible with lectin binding, the sample should be diluted to allow lectin binding. One example of a buffer that can be used in this invention is 25 mM Hepes (pH 8.0), 125 mM NaCl, 5 mM KCl, 1.2 mM KH2PO %, 1.2 mM MgSO %, and 2 mM CaCl.

In the preferred embodiment, lectin molecules are affixed to a solid matrix to form a solid support (as reviewed in Lectins, Biology, Biochemistry, Clinical Biochemistry, Volume 12, which is incorporated by reference). The techniques of protein conjugation or coupling through activated functional groups are particularly applicable (See, for example, Rodwell et al., 1986, and U.S. Pat. No. 4,493,795). The reagent is typically affixed to the solid matrix by adsorption from an aqueous medium although other modes of affixation, well known to those skilled in the art, can be used. Lectins are usually coupled to agarose by cyanogen bromide. Conconavalin A coupled to Sepharose was the first material of this sort to be used and has been widely used in the isolation of polysaccharides and glycoproteins. Other lectins that have been used include lentil lectin, wheat germ agglutinin, leukoagglutinin (L-PHA), and Helix pomatia lectin. The matrix should be a substance that itself does not adsorb glycoproteins or glycans to any significant extent and that has a broad range of chemical, physical and thermal stability. The lectin should be coupled in such a way as to not affect its binding properties. Useful solid matrices are also well known in the art. Such materials are water insoluble and include the cross-linked dextran available under the trademark SEPHADEX from Pharmacia Fine Chemicals (Piscataway, N.J.); agarose; beads of polystyrene about 1 micron to about 5 millimeters in diameter available from Abbott Laboratories of North Chicago, Ill.; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride. In one embodiment, the bound lectin is incubated with glycoproteins and complexes are purified by precipitation of the lectin-bound matrix. In this embodiment, non-bound glycoproteins are removed and precipitates may be washed before elution.

The lectins that can be used in this application include those derived from the group consisting of animal derived, galactose-binding lectins (termed Galectins); Ca-dependent (C-type) animal lectins including sialyl-LewisX recognizing selectins and mannose-specific collectins; glycosaminoglycan binding annexins; plant-derived lectins including concanavalin A, and ricin, and invertebrate lectins such as tachylectins or Xenopus oocyte lectins.

Each lectin recognizes a particular form of carbohydrate and many also depend on the structure of the peptide to which the carbohydrate is attached. Therefore, the choice of lectin will dictate the subpopulation of membrane-anchored glycoproteins that are precipitated. Thus, lectins can be employed that are known to be specific for particular disease states so that an even narrower subset class of glycoproteins is isolated for further analysis.

In addition, naturally occurring glycoproteins will often be produced with a heterogenous range of attached oligosaccharide chains. Due to their binding specificity, a given lectin may not bind all of the oligosaccharide forms present. Therefore, using different lectins will allow a greater application of this technique to identify novel diagnostic glycoproteins. In another embodiment, to increase the likelihood of identifying a serum marker for disease states, this method can be repeated using several immobilized lectins in successive assays. In addition to successive assays, different free lectins could be incubated with immobilized lectins to pre-adsorb the proteins, reducing the total number of proteins that must be analyzed in each assay.

In a preferred embodiment, the lectin used is L-PHA (Phytohemagglutinin-L). PHA, the lectin extract from the red kidney bean (Phaseolus vulgaris), contains potent, cell agglutinating and mitogenic activities. In further preferred embodiments, the lectins used include Concanavalin A (ConA), which has affinity for oligomannosyl saccharides found in N-glycans, galectins including Galectin LEC-6, specific for LacNAc-containing glycans, Aleuria aurantia lectin (AAL) with broad specificity for L-Fuc-containing oligosaccharides, Peanut agglutinin (PNA), which is specific for Galβ1-3GalNAc), found widely in O-glycans, and Helix pomatia lectin (HPA), which binds N-acetylgalactosamine.

Depending on the lectin affinity method employed, bound glycoproteins can be eluted from the immobilized lectin in by any method known in the art. In one embodiment, the bound glycoproteins can be eluted using haptene competition for the binding site of the lectin. The haptene compound used will vary by lectin. In one example, methyl alpha-D-mannopyranoside is used for ConA competition. In another example, glycoproteins bound to a wheat germ agglutinin column are eluted with N-acetyl glucosamine. Elution can also be carried out simply by changing buffer conditions to favor dissociation of the lectin-carbohydrate affinity. Several methods are known in the art to elute proteins, for instance by FPLC using gradients (either step or continuous) of increasing concentrations of competing compounds. One method to monitor release of glycoproteins into collected fractions is to measure the absorbance of the sample at 280 nm. In another embodiment of the invention, the glycoproteins that are absorbed to the immobilized lectin are eluted using a pH gradient.

### Cells/Disease Application

It will be appreciated that the present invention can be used to identify serum markers associated with a wide variety of disease states. In a preferred embodiment the invention will be used to identify markers from diseased cells that display aberrant glycosylation patterns when compared to non-diseased cells.

In another embodiment, the invention provides for a method to identify the glycosylation patterns on recombinant proteins of interest which have been expressed in cells and can therefore be used to identify the glycosyltransferase activity in these cells. Methods to express recombinant proteins in cells of mammalian origin are well known in the art and, in the preferred embodiment, include incorporation of DNA comprising a promoter sequence, a sequence for a protein of interest, a sequence to promote GPI anchoring of said protein to the external leaflet of the plasma membrane (as described), and optionally an affinity tag into the cell. Affinity tags are well known in the art and can be chosen as compatible with specific embodiments of the invention. Methods of incorporating the DNA can include direct transfection as well as infection with a carrier such as an adeno- or retro-virus. Cells that can be used to express recombinant protein can include any mammalian cell including those found in situ, and including both cells harboring a disease and control cells not harboring a disease (as defined herein). In this embodiment, recombinant glycoprotein that is tagged with an affinity tag can be separated from total cleaved proteins by affinity chromatography using a reagent suitable for recognizing said affinity tag.

Suitable tissues from which cells are derived are blood, muscle, nerve, brain, heart, lung, liver, pancreas, spleen, thymus, esophagus, stomach, intestine, kidney, testis, ovary, skin, bone, breast, uterus, bladder, spinal cord, or various kinds of body fluids. The cells may also differ in developmental stage, as well as developmental origin such as ecotodermal, mesodermal, and ectodermal origin.

In one preferred embodiment of the invention, cells from hyperproliferative disorders are used. Examples of hyperproliferative disorders for which disease markers can be investigated include but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvis, skin, soft tissue, spleen, thorax, and urogenital tract, Acute Childhood Lymphoblastic Leukemia; Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphorria, Adult Lymphocytic Leukemia, Adult Non-Hodgkin's Lymphoma, Adult Primary Liver Cancer, Adult Soft Tissue Sarcoma, AIDS-Related Lymphorria, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphorria, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalanic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma. Endometrial Cancer, Ependymoma, Epithelial Cancer, Esophageal Cancer, Ewing's Sarcoma and Related Tumors, Exocrine Pancreatic Cancer, Extraeranial Germ Cell Tumor, Extragonadal Germ Cell Tumor, Extrahepatie Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lympho proliferative Disorders, Macroglobulinemia, Male Breast Cancer, Malignant Mesothelioma, Malignant Thymoma, Medulloblastomia, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyrigeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid, Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethial Cancer, Uterine Cancer, Uterine Sarcoma, Vaginal Cancer, Visual Pathway and Hypothalamic Glioma, Vulvar Cancer, Waldenstroin's Macroglobulinemia, Wilm's Tumor, and any other hyperproliferative disease located in an organ system listed above. Hyperplastic disorders for which diagnostic markers can be developed include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, foca epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

In another embodiment the invention is employed to identify markers of premalignant conditions known or suspected of preceding progression to neoplasia or cancer, in particular, where non-neoplastic cell growth is consisting of hyperplasia, metaplasia, or most particularly, dysplasia has occurred (for review of such abnormal growth conditions, see Robbins. and Angell, 1976).

Additional diseases or conditions for which markers can be investigated using the methods of the present invention include but are not limited to, progression, and/or metastases of malignancies and related disorders such as leukemia (including acute leukemia (e.g., acute lymphocytic leukemia, acute myelocytic leukemia (including myeloblastic, promyelocytic, mylomonocytic, monocytic, and erythroleukemia)) and chronic leukemia (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, Sarcomas and, carcinomas such as fibrosarcoma, myxosarcoma, fiposarcoma, chondrosarcoma, osteogenic sarcoma, chordoma, anglosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendrogliomia, menangioma, melanoma, neuroblastoma, and retinoblastoma.

Although tumors are the best example of diseases in which protein glycosylation is known to be dramatically altered, any other cell for which there is a reason to desire identification of novel proteins which are likely to be released can be used. In another embodiment of the invention, cells taken from patients, or from a model organism harboring diseases other than cancer can also be used to identify differences in serum protein glycosylation. These diseases include muscular dystrophy (Michele et al., 2002; Moore et al., 2002), diabetic microvascular complications, and cystic fibrosis (Scanlin and Glick, 2001).

In another embodiment, cells harboring diseases including autoimmune disorders (such as, multiple sclerosis, Sjogren's syndrome, Hashimoto's thyroiditis, biliary cirrhosis, Behcet's disease, Crohn's disease, polymyosifis, systemic lupus erythematosus and immune-related glomeruionephritis and rheumatoid arthritis) and viral infections (such as herpes viruses, pox viruses and adenoviruses), inflammation, graft v. host disease, acute graft rejection, and chronic graft rejection are used for study.

Diseases associated with increased apoptosis, which are also likely to exhibit specific glycoprotein markers of disease in released proteins include AIDS; neurodegenerative disorders (such as Alzheimer's disease, Parkinson's disease, amyotrophic lateral sclerosis, retinitis pigmentosa, cerebral degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemiclupus erythematosus and immune-related glomerulonephritis and rheumatoid arthritis) myelodysplastic syndromes (such as a plastic anemia), graft Y host disease, ischemic injury (such as that caused by myocardial infarction, stroke and repercussion injury), liver injury (e.g., hepatitis related liver injury, ischemia/Eeperfusion injury, cholestosis (bile duct injury) and liver cancer); toxin-induced liver disease (such as that caused by alcohol), septic shock, cachexia and anorexia.

In addition, because immortal mammalian tumor cell lines recapitulate many of the features of the parent cells they were developed from, including regulated release of proteins, one embodiment of this invention analyzes immortalized tumor cell lines for potentially released glycoproteins. In addition, this method can be applied to hyperproliferative disorders (described above), which have been exposed to chemotherapeutic agents. In another embodiment, the disorders associated with increased apoptosis (such as AIDS; neurodegenerative disorders; autoimmune disorders, myelodysplastic syndromes, graft Y host disease, ischemic injury, liver injury, toxin-induced liver disease, septic shock, and cachexia) can be analyzed after treatment. These embodiments have the potential to identify serum markers indicating relapse or remission in patients who have undergone treatment.

In one aspect of the invention, the glycoproteins that are likely to be released into serum are separated from different samples of tissue harboring disease at different stages. This provides for a method to identify prognosis markers of the individual disease. Two disease stages may have a particular protein similarly expressed and glycosylated, however the evaluation of a number of proteins simultaneously allows the generation of a profile that is unique to the state of the cell.

### 6. Glycosylation Profiles

In one embodiment of the invention, the glycosylation patterns of the released glycoproteins are analyzed to identify aberrations caused by the disease. This analysis is preferably preceded by a further separation of the glycoproteins, so that the glycosylation pattern can be associated with a particular glycoprotein or band of glycoproteins.

In one embodiment, after purification, the sample containing the potentially secreted or released glycoproteins is subjected to a separation step according to one physical or chemical property, for example, the proteins are subjected to SDS-PAGE. The glycoproteins are immobilized to allow identification, for example on a membrane, preferably a polyvinyl difluoride membrane (PVDF). PVDF-based membranes have higher protein-binding capacity and result in better average initial and repetitive yields. Preferably the immobilized proteins will be retrievable from the array. The glycoproteins are then visualized by a specific binding agent to yield a glycoprotein expression pattern. A “specific binding agent” is a molecular entity capable of selectively binding a reagent species (such as a subset of sugar moieties) or a complex containing such a species. Exemplary specific binding agents are conjugated lectins, antibody molecules, complement proteins or fragments thereof, and the like. In preferred embodiments, the specific binding agent is labeled. However, when the specific binding agent is not labeled, the agent can be used as an amplifying means or reagent.

In another embodiment, after separation, the sample containing the potentially secreted or released glycoproteins of interest is subjected to two successive separation steps. In the first separation step, the glycoproteins are separated according to one physical or chemical property, for example, proteins are separated by isoelectric focusing along a first axis. In the second separation step, the glycoproteins in this one-dimensional array are separated according to a second physical or chemical characteristic, for example, proteins separated by isoelectric focusing are subjected to SDS-PAGE along a second axis perpendicular to the first axis. The glycoproteins in the array are immobilized to allow identification, for example on a PVDF-based membrane. The glycoproteins are then visualized by a specific binding agent to yield a glycoprotein expression pattern. Preferably the immobilized proteins will be retrievable from the array.

In one embodiment, a glycosylation profile is developed via lectin affinity to the separated glycoproteins. In this embodiment, the glycoproteins are sequentially or simultaneously probed with a plurality of lectins chemically coupled to an indicating group, to identify a lectin binding pattern for the glycoproteins. The indicating group is preferably a compound that can be detected by luminescence and this is detected in each sample of the array to form an staining pattern representative of the differential glycoprotein profile of the cells. “Luminescence” is the term commonly used to refer to the emission of light from a substance for any reason other than a rise in its temperature. Luminescence which results from a chemical reaction is usually referred to as “chemiluminescence”. If photoluminescence is the result of a spin-allowed transition (e.g., a single-singlet transition, triplet-triplet transition), the photoluminescence process is usually referred to as “fluorescence”. A “luminescent label” may have any of the above-described properties.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that an immunoreactant has formed. In a further embodiment, the indicating group is a fluorophore, in which case the direct fluorescence of the compound can be detected. The linking of labels, i.e., labeling of, polypeptides and proteins is well known in the art.

This invention also provides a method to distinguish the differentially expressed Or glycosylated cleaved glycoproteins by: (a) preparing a first cleaved glycoprotein expression pattern from the separated cleaved glycoproteins; and (b) subtracting from the first cleaved glycoprotein expression pattern a second glycoprotein expression pattern, wherein said second glycoprotein expression pattern is from cells not harboring the disease. In this embodiment, the second glycoprotein expression pattern is prepared by: (a) providing a plurality of non-diseased control cells not harboring the disease; (b) incubating the glycoproteins with a phospholipase to yield cleaved glycoproteins and uncleaved glycoproteins; and (c) separating said cleaved glycoproteins from said uncleaved glycoproteins.

Normal tissue may thus be distinguished from diseased tissue, and within diseased tissue, different prognosis states may be determined by differential glycosylation. “Differential glycosylation” refers to an increased (upregulated or present) or decreased (downregulated or absent), glycosylation of glycoproteins in diseased versus normal tissue or cells. Additionally, this invention provides means for identifying glycoproteins that are differentially expressed in disease versus normal tissue or cells. “Differential expression” refers to an increased or decreased protein expression in a sample. By comparing glycosylation profiles information regarding glycosylation levels in each of these states is obtained. Levels of glycosylation of different glycoproteins in diseased cells can be evaluated for diagnostic and prognostic purposes or to screen candidate agents. In a preferred embodiment, the diseased cells are from humans; however, as will be appreciated by those in the art, profiles from other organisms may be useful in animal models of disease and drug evaluation; thus, other profiles are provided, from vertebrates, including mammals, including rodents (rats, mice, hamsters, guinea pigs, etc.), primates, farm animals (including sheep, goats, pigs, cows, horses, etc). Profiles from other organisms may be obtained using the techniques outlined.

In an additional embodiment, this invention provides a method of detecting differential expression of a target protein in a multiplicity of cell types derived from at least two subjects. Such method involves: (a) staining a first glycoprotein array with a compound reactive to either the general characteristics of glycoproteins in the array (such as a lectin) or specific for a subset of glycoproteins (such as an antibody reactive to one or more proteins within the sample), chemically coupled to a compound that can be detected; (b) detecting the reactive compound in the array that forms a first staining pattern representative of the differential expression of said target in the multiple types of cells of the first subject; (c) staining a second glycoprotein array from a second subject with the same compound; (d) detecting the stain in the second array to form a second staining pattern representative of the differential glycoproteins in the second subject; and (e) comparing the patterns, thereby detecting the differential expression or glycosylation of proteins in the subjects.

To generate a measurable glycosylation profile or glycoprotein expression pattern, several techniques can be used. In one embodiment, the array is imaged with a detector to generate a set of coordinates and a signal value for each point in the sample. This can be accomplished on a one-dimensional or two-dimensional array. Analysis of the coordinates is performed, resulting in a profile that represents the relative abundance of each glycoproteins and its attributes as deduced from its coordinates in the two-dimensional array. For example, a profile derived from imaging a gel containing proteins separated by isoelectric focusing followed by SDS-PAGE represents the isoelectric point (p1), apparent molecular weight (MW) and relative abundance of a plurality of detected proteins. The glycoprotein profiles can then be used to compare the glycosylation state of glycoproteins in each sample. In one embodiment, a first set of profiles is compared with a second set of profiles to identify glycoproteins that are represented in the first set (or in a percentage of the first set) and are absent from the second set (or are absent from percentage of the second set). In other embodiments, sets of profiles are compared to identify glycoproteins that are present at higher levels of expression or glycosylation one sample set than in another sample set. These analyses can be accomplished using a computer, as is described in detail in U.S. Pat. No. 6,459,994.

### 7. Identification of Specific Glycoproteins

Several general techniques can be used to identify the specific glycoproteins with a high probability of being released into serum for potential use as disease diagnostic markers, including microsequencing, amino acid composition analysis, or mass spectrometry analysis.

In one embodiment, differentially expressed or glycosylated, separated glycoproteins, or differentially expressed or glycosylated, cleaved and separated glycoproteins, are identified by proteolysis and liquid chromatography/mass spectrometry (LC/MS). Mass spectrometry is an established analytical technique that identifies compounds by the mass (more correctly, mass to charge ratio) of the analyte molecule. Mass spectrometry is especially noteworthy among analytical techniques because the signals produced by a spectrometer are the direct result of chemical reactions such as ionization and fragmentation, rather than energy state changes that are typical of most other spectroscopic techniques. Samples are digested before subjecting them to MS. Preferably, the N-termini of the peptide fragments are free, i.e, the N-terminal end of each peptide is a free amino group. One preferred reagent for the protein digestion is cyanogen bromide (CNBr). As will be recognized by one skilled in the art, the conditions of the digest are adjusted such that peptides are produced which are amenable for separation, detection and identification (see Protein Structure: Practical Approach, T. E. Creighton, ed., 1989, incorporated herein by reference). In another preferred embodiment of the invention, the purified glycoproteins are sequentially subjected to proteolysis by trypsin.

To increase resolution, additional separation steps, including chromatography separation methods based on physical parameters such as molecular weight, charge, or hydrophobicity can be used. Preferred chromatography methods include high pressure liquid chromatography (HPLC) and automated liquid chromatography (FPLC). LC-MS is limited in its application because a single mass separation device has limited selectivity and struggles to detect lower concentration proteins in the presence of more abundant ones. This limitation has led to the development of systems with tandem mass spectrometers (LC-MS-MS and electrospray MS-MS), in which multiple mass-resolving devices are used to identify complex mixtures. The peptides may also be analyzed by electrospray (ESI) or matrix-assisted laser desorption ionization (MALDI) MS. Both ESI and MALDI are effective means of producing gas phase ions of proteins, peptides and other biomolecules for MS analysis. ESI sources typically are used on quadrupole or ion trap mass analyzers, whereas MALDI sources are typically used with time-of-flight (TOF) mass analyzers. Although a variety of hybrid instruments have been produced. Both ESI and MALDI are capable of sub-femtomole sensitivity for peptide analysis. ESI-triple quadrupole or ESI-ion trap instruments can be used for MS-MS analyses that yield peptide sequence information. MALDI instruments equipped with post source decay capability also can generate peptide sequence information although ESI-triple quadrupole and ESI-ion trap instruments are considered the best for true MS-MS sequencing (The techniques used to identify peptide and compare peptides are reviewed in U.S. Pat. No. 6,379,970, which is hereby incorporated by reference).

In one embodiment, a subset of proteins from a glycosylation profile is isolated and sequenced. One or more biomolecules so identified are selected for isolation. In one embodiment, this selection is made automatically by a computer, in accordance with pre-ordained programmed criteria. In another embodiment, a human operator reviews the results and selects the protein targets for isolation. Spots containing the proteins of interest typically are excised from gels and subjected to proteolytic digestion. The proteins can be visualized on the blot by staining with Coomassie blue, Ponceau S, or Amido Black (the latter two are less sensitive stains). Coomassie blue is sensitive enough to detect 50-200 ng of protein on PVDF. The size of an excised piece of PVDF membrane containing the protein band should be smaller than 40 mm². If necessary, a standard protein can be blotted on the PVDF to verify that the protein was electroblotted efficiently and that the N-terminus was not blocked during the SDS-PAGE and blotting procedures. Contaminating salts can be removed by washing the membrane with deionized water, prior to sequencing the sample on PVDF membrane. The samples can also be desalted using any other conventional technique including desalting on an ion exchange resin.

In addition, a selected glycoprotein can be analyzed to determine its full or partial amino acid sequence, to detect and characterize any associated oligosaccharide moieties, and to study other aspects of post-translational processing, e.g. phosphorylation, myristylation and the like.

In addition, the invention encompasses computer-implemented methods for detecting differential expression of a target glycoprotein in a multiplicity of cell types. Also included are computer-based systems for detecting differential expression of a target glycoprotein in a multiplicity of cell types derived from at least two subjects.

In one example, separated glycoprotein can be sequenced directly by automated Edman degradation with a model 470A Applied Biosystems gas phase sequencer equipped with a 120A PTH amino acid analyzer or sequenced after digestion with chemicals or enzymes, including proteases, preferably trypsin. PTH amino acids can be integrated using a ChromPerfect data system (Justice Innovations, Palo Alto, Calif.). Sequence interpretation can be performed on a VAX 11/785 Digital Equipment Corporation computer as described by Henzel et al., 1987. In some cases, aliquots of the HPLC fractions are separated by SDS-PAGE, transferred to a membrane and stained. The specific protein is then excised from the blot for N-terminal sequencing. To determine the internal protein sequences, HPLC fractions can be dried under vacuum, resuspended in appropriate buffers, and digested. After digestion, the resultant peptides are sequenced as a mixture or are resolved by HPLC on a C4 column developed with a propanol gradient in 0.1% TFA before sequencing as described above. to avoid high glycine background during Edman degradation, electroblotting can be performed in 10 mM CAPS (3-cyclohexylamino-1-propanesulfonic acid) buffer with 10% methanol, pH 11.0 for at least 15-20 minutes. Following the transfer, the membrane is rinsed with deionized water to reduce Tris and glycine contaminants.

The invention further provides for identification of proteins after sequencing by database searching. Proteins amino acid sequences are available in public databases such as ChemicalAbstracts Services Databases (e.g., the CAS Registry), GenBank, and GenSeq and can be found at the Center for Biotechnology Information (NCBI) webpage at www.ncbi.nlm.nih.gov. To identify proteins after sequencing, the amino acid sequence is compared to the available sequences using a sequence comparison program. These include programs such as BLAST, available from the NCBI. BLAST (Basic Local Alignment Search Tool) is a set of similarity search programs designed to explore all of the available sequence databases regardless of whether the query is protein or DNA. The scores assigned in a BLAST search have a well-defined statistical interpretation, making real matches easier to distinguish from random background hits (Altschul et al., 1990). For a discussion of basic issues in similarity searching of sequence databases, see Altschul et al., 1994 which is fully incorporated by reference.

In a further embodiment of the invention, secreted proteins from Triton X-114 lysed cells are further identified by recognition of their signal sequence. Many eukaryotic proteins normally secreted from the cell contain an endogenous signal sequence as part of the amino acid sequence. This sequence targets the protein for export from the cell via the endoplasmic reticulum and Golgi apparatus. The signal sequence is typically located at the amino terminus of the protein, and ranges in length from about 13 to about 36 amino acids. Although the actual sequence varies among proteins, all known eukaryotic signal sequences contain at least one positively charged residue and a highly hydrophobic stretch of 10-15 amino acids (usually rich in the amino acids leucine, isoleucine, alanine, valine and phenylalanine) near the center of the signal sequence. The signal sequence is normally absent from the mature form of the protein, as it is cleaved by a signal peptidase located on the endoplasmic reticulum during translocation of the protein into the endoplasmic reticulum. The protein with its signal sequence still attached is often referred to as the ‘pre-protein’ or the immature form of the protein. However, not all secreted proteins contain an amino terminal signal sequence that is cleaved. Some proteins, such as ovalbumin, contain a signal sequence that is located on an internal region of the protein. This sequence is not normally cleaved during translocation.

One aspect of this invention is to allow targeting of diseased tissue by cytotoxic agents. This could drastically reduce the incidental side effects and enhance the efficacy of known compounds. Heavy metal ions, for example, can be linked to molecules that recognize disease markers identified using the preceding techniques. Because the glycoprotein markers of disease are specific they may be used to enhance the specificity of targeted chemotherapies. For example, a compound such as an antibody, may recognize both the glycosylation profile and peptide sequence of a glycoprotein disease marker. Therefore, the compound will bind to cells expressing this combined phenotype, i.e. the diseased cells. The cytotoxic agent will therefore preferentially accumulate in diseased tissue and be excluded from non-diseased tissue. Targeted therapies, for example the coupling of a cytotoxic agent to a monoclonal antibody recognizing previously described disease markers is already known in the art. For example, techniques are described in U.S. Pat. No. 5,663,306 or Glassy and Dillman, 1988, Molecular Biotherapy. 1(1):7-13 which are incorporated herewith by reference. Cytotoxic agents can include, but are not limited to, heavy metal ions, plant or insect toxins, or venoms. Agents can also include chemicals designed to interfere with protein-protein interactions or the enzymatic or catalytic capacity of a cellular protein. Agents can also damage DNA (such as cisplatin) or disrupt the ionic gradients in cells by, for example, chelating divalent cations. Methods to conjugate the therapeutic compound to the glycoprotein recognition agent will differ, depending on the nature of both the therapeutic compound and the recognition agent. Conjugation methods are known in the art and include the addition of a linker molecule to the recognition agent such that this linker can be conjugated to a set of therapeutic agents. In addition, the recognition agent can be comprised of multiple molecules, such as several antibodies or oligonucleotides. After conjugation of the therapeutic agent to the glycoprotein recognition agent, the conjugate can be administered to a patient by injection, ingestion, or any other means available.

### DEFINITIONS

In this specification, “glycoprotein” refers to any protein in which one or more carbohydrate units have been attached covalently to the protein by posttranslational processing.

In this specification, “non-glycoprotein” refers to any protein that is not glycosylated in its native state.

In this specification, the phrase “cleaved glycoprotein” refers to a glycoprotein that has been cleaved from a cellular membrane by the activity of a phospholipase. These include all glycoproteins that were attached to the membrane by virtue of phosphodiester bond, via a GPI anchor.

In this specification, “detergent” is defined as a surface-active agent (surfactants) containing a hydrophobic portion, which is more soluble in oil-like solutions, and a hydrophilic portion, which is soluble in water. “Cloud point” is defined as the temperature at which solutions of these detergents undergo phase separation, thought to occur because of dehydration oxyethylene head groups and consequent formation of giant micelles

“Differential glycosylation” refers to an increased, upregulated or present, or decreased, downregulated or absent, glycosylation of proteins glycoproteins in diseased versus normal tissue or cells. “Differential expression” refers to an increased or decreased protein expression in a sample.

“Glycoprotein expression pattern” is defined as the pattern of glycoproteins purified through the methods described in this invention.

A “Glycosylation profile” is the normalized analysis of the glycoprotein expression pattern, which can be read through a computer generated program and can be compared to other glycosylation profiles.

The present invention may be embodied in other specific forms without departing from the spirit or essential attributes thereof and accordingly reference should be made to the appended claims rather than the foregoing specifications as indicating the scope of the invention.

### REFERENCES

U.S. Pat. No. 6,261,791, Jul. 17, 2001 Reiter, et al. Method for diagnosing cancer using specific PSCA antibodies
U.S. Pat. No. 5,268,272 Dec. 7, 1993 Mullner, et al. Complexes containing glycosyl-phosphatidylinositol proteins and cholanic acid derivatives, a process for their preparation and their use
U.S. Pat. No. 6,455,494 Sep. 24, 2002 Jefferies, et al. Use of p97 and iron binding proteins as diagnostic and therapeutic agents
U.S. Pat. No. 4,493,795 Jan. 15, 1985 Nestor, Jr., et al. Synthetic peptide sequences useful in biological and pharmaceutical applications and methods of manufacture
U.S. Pat. No. 6,459,994. Oct. 1, 2002 Parekh, et al. Methods for computer-assisted isolation of proteins
U.S. Pat. No. 6,379,970 Apr. 30, 2002 Liebler, et al. Analysis of differential protein expression
U.S. Pat. No. 5,753,462: May 19, 1998 Lok, Si; Secretion leader trap cloning method
U.S. patent application 20020058288A1 May 16, 2002 Koolen, M. J. M, et al. Toxoplasma Gondii Glycoconjugates.
Japanese patent document JP 55034039,
Altschul S F, Gish W, Miller W, Myers E W, Lipman D J. (1990) Basic local alignment search tool J Mol Biol 215(3):403-10.
Altschul S F, Boguski M S, Gish W, Wootton J C (1994) Issues in searching molecular sequence databases. Nat Genet. 6(2):119-29.
Brooks S A. Leathem A J. (1991) Prediction of lymph node involvement in breast cancer by detection of altered glycosylation in the primary tumour. Lancet. 338(8759):71-4.
Brooks, S. A. & Leathem, A. J. C. (1995). Expression of GalNAc glycoproteins by breast cancers. British Journal of Cancer 71, 1033-1038.
Brooks S A. Lymboura M. Schumacher U. Leathem A J. (1996) Histochemistry to detect Helix pomatia lectin binding in breast cancer: methodology makes a difference. Journal of Histochemistry & Cytochemistry. 44(5):519-24.
Deeg M A, Davitz M A. (1995) Glycosylphosphatidylinositol-phospholipase D: a tool for glycosylphosphatidylinositol structural analysis. Methods in Enzymology 250:630-640.
Deeg M A, Bierman E L, Cheung M C. (2001) GPI-specific phospholipase D associates with an apoA-I- and apoA-IV-containing complex. J Lipid Res. 42(3):442-51.
Dwek, M. V., Brooks, S. A., Streets, A. J., Harvey, D. J. & Leathem A. J. C. (1996) Oligosaccharide release from frozen and paraffin wax embedded archival tissues. Analytical Biochemistry 242, 8-14.
Dwek M V, Ross H A, Leathem A J C (2001) Proteome and glycosylation mapping identifies post-translational modifications associated with aggressive breast cancer Proteomics 1:p756-762.
Endo T. (1996) Fractionation of glycoprotein-derived oligosaccharides by affinity chromatography using immobilized lectin columns Journal of Chromatography. A 720(1-2):251-61.
Essen L O, Perisic O, Cheung R, Katan M, Williams R L. (1996) Crystal structure of a mammalian phosphoinositide-specific phospholipase C delta Nature. 380(6575):595-602.
Essen L O, Perisic O, Lynch D E, Katan M, Williams R L. (1997) A ternary metal binding site in the C2 domain of phosphoinositide-specific phospholipase C-delta1. Biochemistry. 36(10):2753-62.
Ferguson M A and Williams A F. (1988) “Cell-Surface Anchoring of Proteins via Glycosyl-Phosphatidylinositol Structures,” Ann. Rev. Biochem. 57:285-320.
Glassy M C. Dillman R O. (1988) “Molecular biotherapy with human monoclonal antibodies.” Molecular Biotherapy. 1(1):7-13.
Gombos G. Reeber A. Zanetta J P. Vincendon G. (1976) Fractionation of nervous tissue membranes glycoproteins. pp. 829-44. (Monograph Citation).
Hakomori S. (1996) Tumor malignancy defined by aberrant glycosylation and sphingo(glyco)lipid metabolism Cancer Research. 56(23):5309-18
Hanisch F G. (2001) O-glycosylation of the mucin type. Biological Chemistry. 382(2):143-9.
Hakomori S. (2002) Glycosylation defining cancer malignancy: new wine in an old bottle. Proceedings of the National Academy of Sciences of the United States of America. 99(16):10231-3.
Harvey D J. (2001) Identification of protein-bound carbohydrates by mass spectrometry. Proteomics. 1(2): 311-28.
Henner D J. Yang M. Chen E. Hellmiss R. Rodriguez H. Low M G. (1988) Sequence of the Bacillus thuringiensis phosphatidylinositol specific phospholipase C. Nucleic Acids Research. 16(21):10383.
Henzel W J. Rodriguez H. Watanabe C. (1987) Computer analysis of automated Edman degradation and amino acid analysis data. Journal of Chromatography. A. 404(1):41-52
Hetland O. and Prydz H. (1982) Phospholipase C from Bacillus cereus has sphingomyelinase activity. Scandinavian Journal of Clinical & Laboratory Investigation. 42(1):57-61.
Hoja-Lukowicz D. Litynska A. Wojczyk B S. (2001) Affinity chromatography of branched oligosaccharides in rat liver beta-glucuronidase. Journal of Chromatography. B, Biomedical Sciences & Applications. 755(1-2):173-83.
Ikezawa H. Yamanegi M. Taguchi R. Miyashita T. Ohyabu T. (1976) Studies on phosphatidylinositol phosphodiesterase (phospholipase C type) of Bacillus cereus. I. purification, properties and phosphatase-releasing activity. Biochimica et Biophysica Acta. 450(2):154-64.
Jaques A J. Opdenakker G. Rademacher T W. Dwek R A. Zamze S E. (1996) The glycosylation of Bowes melanoma tissue plasminogen activator: lectin mapping, reaction with anti-L2/HNK-1 antibodies and the presence of sulphated/glucuronic acid containing glycans. Biochemical Journal. 316 (2):427-37.
Kim Y J. Varki A. (1997) Perspectives on the significance of altered glycosylation of glycoproteins in cancer. Glycoconjugate Journal 14(5): 569-576; January 1997
Kishino S. Miyazaki K. (1997) Separation methods for glycoprotein analysis and preparation. Journal of Chromatography. B, Biomedical Sciences & Applications. 699(1-2):371-81.
Koke J A. Yang M. Henner D J. Volwerk J J. Griffith O H. (1991) High-level expression in Escherichia coli and rapid purification of phosphatidylinositol-specific phospholipase C from Bacillus cereus and Bacillus thuringiensis Protein Expression & Purification. 2(1):51-8.
Kuppe A. Evans L M. McMillen D A. Griffith O H. (1989) Phosphatidylinositol-specific phospholipase C of Bacillus cereus: cloning, sequencing, and relationship to other phospholipases Journal of Bacteriology. 171(11): 6077-83
Leathem A J. Brooks S A. (1987) Predictive value of lectin binding on breast-cancer recurrence and survival. [Journal Article] Lancet. 1(8541):1054-6
Lectins, Biology, Biochemistry, Clinical Biochemistry, Volume 12, including Proceedings from the 17th International Lectin Meeting in Würzburg, (1997) ed. van Driessche E, Beeckmans S, and Bøg-Hansen T C, published by TEXTOP, Lemchesvej 11, DK-2900 Hellerup, Denmark
Li J Y, Hollfelder K, Huang K S, Low M G (1994) Structural features of GPI-specific phospholipase D revealed by proteolytic fragmentation and Ca2+ binding studies. J Biol Chem 269(46):28963-71
Low M G. Finean J B. (1977) Modification of erythrocyte membranes by a purified phosphatidylinositol-specific phospholipase C (Staphylococcus aureus). Biochemical Journal. 162(2):235-40.
Low M G and Prasad A R. (1988) A phospholipase D specific for the phosphatidylinositol anchor of cell-surface proteins is abundant in plasma. Proceedings of the National Academy of Sciences of the United States of America. 85(4):980-984.
Low M G. (1990) “Degradation of glycosyl-phosphatidylinositol anchors by specific phospholipases”, in Molecular and Cell Biology of Membrane Proteins: Glycolipid Anchors of Cell-structure Proteins, A. J. Turner (ed.), Ellis Horwood, New York. Chapter 2, pp 35-63
Madore N. Smith K L. Graham C H. Jen A. Brady K. Hall S. Morris R. (1999) Functionally different GPI proteins are organized in different domains on the neuronal surface. EMBO Journal. 18(24):6917-26.
Meri S, Lehto T, Sutton C W, Tyynela J, Baumann M. (1996) Structural composition and functional characterization of soluble CD59: heterogeneity of the oligosaccharide and glycophosphoinositol (GPI) anchor revealed by laser-desorption mass spectrometric analysis. Biochem. J. 316:923-935.
Michele D E, Barresi R., Kanagawa M, Saito F., Cohn R. D., Satz J. S., Dollar J., Nishino I., Kelley R. I., Somer H., Straub V., Mathews K. D., Moore S. A., Campbell K. P. (2002) Post-translational disruption of dystroglycan-ligand interactions in congenital muscular dystrophies Nature 418, 417-421.
Moore S. A., Saito F., Chen J., Michele D. E., Henry M. D., Messing A., Cohn R. D., Ross-Barta S. E., Westra S., Williamson R. A., Hoshi T., Campbell K. P. (2002) Deletion of brain dystroglycan recapitulates aspects of congenital muscular dystrophy Nature 418, 422-425
Park S W, Kang B Y, Yoon H J, Park E M, Choi K, Lee H E, Hooper N M, Park H S. (2002) Spontaneous release of glycosylphosphatidylinositol (GPO-anchored renal dipeptidase from porcine renal proximal tubules. Archives of Pharmacal Research 25(1):80-85.
Park S W, Choi K, Lee H B, Park S K, Turner A J, Hooper N M, Park H S. (2002) Glycosyl-phosphatidylinositol (GPI)-anchored renal dipeptidase is released by a phospholipase C in vivo. Kidney Blood Press Res; 25(1):7-12.
Park S W, Choi K, Kim I C, Lee H H B, Hooper N M, Park H S Endogenous glycosylphosphatidylinositol-specific phospholipase C releases renal dipeptidase from kidney proximal tubules in vitro Biochem. J. 353:339-344.
Protein Structure: Practical Approach, T. E. Creighton, ed., ILR Press, 1989, pp 117-144,
Przybyo M, Hoja-Lukowicz D, Lityñska A, Laidler P. (2002) Different glycosylation of cadherins from human bladder non-malignant and cancer cell lines 2. Cancer Cell International 2:6
Rodwell J D. Alvarez V L. Lee C. Lopes A D. Goers J W. King H D. Powsner H J. McKearn T J. (1986) Site-specific covalent modification of monoclonal antibodies: in vitro and in vivo evaluations. Proceedings of the National Academy of Sciences of the United States of America. 83(8): 2632-6
Robbins. and Angell (1976) Basic Pathology, 2d Ed. W. B. Saunders Co., Philadelphia, pp. 68-79
Scanlin T. F, and Glick M. C. (2001) Glycosylation and the cystic fibrosis transmembrane conductance regulator Respir Res 2001 2: 276-279
Schumacher D U. Randall C J. Ramsay A D. Schumacher U. (1996) Is the binding of the lectin Helix pomatia agglutinin (HPA) of prognostic relevance in tumours of the upper aerodigestive tract?. European Journal of Surgical Oncology. 22(6):618-20.
Slein M W. Logan G F Jr. (1967) Lysis of Escherichia coli by ethylenediaminetetraacetate and phospholipases as measured by beta-galactosidase activity. [Journal Article] Journal of Bacteriology. 94(4):934-41.
Springer G F. Desai P R. Banatwala I. (1975) Blood group MN antigens and precursors in normal and malignant human breast glandular tissue. Journal of the National Cancer Institute. 54(2):335-9.
Srivastava V, Gray G R. (1993) Structural analysis of sialic acid-containing carbohydrates by the reductive-cleavage method. Carbohydr Res. 248:167-78
Sumi S. Arai K. Kitahara S. Yoshida K. (1999) Serial lectin affinity chromatography demonstrates altered asparagine-linked sugar-chain structures of prostate-specific antigen in human prostate carcinoma. Journal of Chromatography. B, Biomedical Sciences & Applications. 727(1-2):9-14.
Taguchi R. Ikezawa H. (1978) Phosphatidyl inositol-specific phospholipase C from Clostridium novyi type A. Archives of Biochemistry & Biophysics. 186(1):196-201
Volwerk J J. Koke J A. Wetherwax P B. Griffith O H. (1989) Functional characteristics of phosphatidylinositol-specific phospholipases C from Bacillus cereus and Bacillus thuringiensis. FEMS Microbiology Letters. 52(3):237-41
Yoshida K I. Honda M. Arai K. Hosoya Y. Moriguchi H. Sumi S. Ueda Y. Kitahara S. (1997) Serial lectin affinity chromatography with concavalin A and wheat germ agglutinin demonstrates altered asparagine-linked sugar-chain structures of prostatic acid phosphatase in human prostate carcinoma. Journal of Chromatography. B, Biomedical Sciences & Applications. 695(2):439-43.
Yoshida K. Moriguchi H. Sumi S. Horimi H. Kitahara S. Umeda H. Ueda Y. (1999) Alterations of asparagine-linked sugar chains of N-acetyl beta-D-hexosaminidase during human renal oncogenesis: a preliminary study using serial lectin affinity chromatography. Journal of Chromatography. B, Biomedical Sciences & Applications. 723(1-2):75-80.

We claim:

1) A method of isolating serum glycoprotein markers that are associated with a disease state comprising:
(a) providing a plurality of cells harboring a disease and comprising glycoproteins;
(b) incubating said cells with an enzyme that cleaves phospholipid anchors to yield cleaved glycoproteins that comprise an inositol moiety and uncleaved glycoproteins;
(c) separating said cleaved glycoproteins from said uncleaved glycoproteins; and
(d) optionally characterizing a glycosylation pattern on one or more of said cleaved glycoproteins.

2) The method of claim 1 wherein the glycosylation pattern is characterized by contacting said cleaved glycoproteins with one or more lectins and determining whether or not the one or more lectins bind said cleaved glycoproteins.

3) The method of claim 1 wherein the diseased cells are incubated in a first buffer before incubation with a phospholipase.

4) The method of claim 3 wherein the first buffer comprises at least one chaotropic agent.

5) The method of claim 3 wherein the first buffer comprises at least one detergent.

6) A method as in claim 5 additionally comprising, before phospholipase cleavage, separating GPI-anchored glycoproteins that have an uncleaved signal sequence from said cells that have been incubated with a first buffer comprising a detergent with a compound comprising an affinity for a GPI-anchor signal sequence.

7) The method of any of claims 1-6 further comprising separating glycoproteins from non-glycoproteins in said cells.

8) The method of claim 7 wherein said glycoproteins are separated from said non-glycoproteins by a process comprising:
(a) contacting said glycoproteins with an immobilized lectin; and
(b) eluting bound glycoprotein off said immobilized lectin, wherein said lectin is optionally specific for the disease.

9) The method of any of claims 1-8 further comprising detecting from said separated cleaved glycoproteins a subset of cleaved glycoproteins that are differentially expressed or glycosylated in cells harboring the disease.

10) The method of claim 9 wherein the differentially expressed or glycosylated cleaved glycoproteins are distinguished by:
(a) preparing a first cleaved glycoprotein expression pattern from the separated cleaved glycoproteins; and
(b) subtracting from the first cleaved glycoprotein expression pattern a second glycoprotein expression pattern, wherein said second glycoprotein expression pattern is from cells not harboring the disease.

11) The method of claim 9 wherein said second glycoprotein expression pattern is prepared by:
(a) providing a plurality of non-diseased control cells not harboring the disease;
(b) incubating the glycoproteins with a phospholipase to yield cleaved glycoproteins and uncleaved glycoproteins; and
(c) separating said cleaved glycoproteins from said uncleaved glycoproteins.

12) The method of claim 11 wherein the non-diseased cells are incubated in a first buffer before incubation with a phospholipase.

13) The method of claim 11 wherein the first buffer comprises at least one detergent.

14) The method of any of claims 10-13 further comprising separating glycoproteins from non-glycoproteins in the non-diseased cells.

15) The method of any of claims 1-14 wherein the diseased cells comprise breast cancer cells.

16) The method of any of claims 1-14 wherein the diseased cells comprise colorectal cancer cells.

17) The method of any of claims 1-14 wherein the diseased cells comprise prostate cancer cells.

18) The method of any of claims 1-14 wherein the diseased cells comprise choriocarcinoma cells.

19) The method of any of claims 1-14 wherein the cells comprise lymphoma cells.

20) The method of any of claims 1-14 wherein the cells comprise pancreatic cancer cells.

21) The method of any of claims 1-14 wherein the cells comprise lung cancer cells

22) The method of any of claims 1-14 wherein the cells comprise bladder cancer cells

23) The method of any of claims 1-9 wherein the disease is cancer.

24) The method of any of claim 3-10 or 12-23 further comprising removing non-solubilized material from said diseased or non-diseased cells after incubation with a first buffer.

25) The method of any of claims 1-23 further comprising identifying said separated, cleaved glycoproteins.

26) The method of any of claims 1-24 wherein the enzyme that cleaves phospholipid anchors is phospholipase C.

27) The method of any of claims 1-26 wherein the cleaved glycoproteins are separated from non-cleaved glycoproteins by immunoprecipitation with an antibody to the inositol moiety.

28) The method of any of claims 1-26 wherein the cleaved glycoproteins are separated from non-cleaved glycoproteins by affinity chromatography of their inositol moiety.

29) The method of any of claims 1-28 wherein said cleaved glycoproteins are identified by proteolysis and liquid chromatography/mass spectrometry (LC/MS).

30) A method of isolating serum glycoprotein markers that are associated with a disease state comprising:
(a) providing a plurality of cells harboring a disease and comprising glycoproteins and non-glycoproteins;
(b) solubilizing said cells in a liquid medium comprising water and a detergent with a cloud point;
(c) separating said liquid medium into an aqueous phase comprising hydrophilic proteins and a detergent phase comprising lipophilic proteins;
(d) separating glycoproteins from non-glycoproteins in said aqueous phase and said detergent phase; and
(e) optionally characterizing a glycosylation pattern on one or more of said separated glycoproteins.

31) A method of claim 30 wherein the detergent comprises a critical micellar temperature, and said solubilized cells are separated into an aqueous phase and a detergent phase by heating to a temperature above the critical micellar temperature.

32) A method of claim 30 or 31 in which the detergent has a cloud point below about 50° C.

33) A method of claim 30 or 31 in which the detergent is Triton X-114.

34) A method of claim 30 further comprising:
(f) contacting said detergent phase with a phospholipase to yield cleaved glycoproteins that comprise an inositol moiety and uncleaved glycoproteins;
(g) separating the uncleaved glycoproteins from the cleaved glycoproteins; and
(h) optionally characterizing a glycosylation pattern on one or more of said detergent phase, uncleaved glycoproteins.

35) The method of claim 30 in which said glycoproteins are identified by proteolysis and liquid chromatography/mass spectrometry (LC/MS).

36) The method in any of claims 30-35 in which said glycoproteins are separated from non-glycoproteins by a method comprising:
(a) contacting hydrophilic glycoproteins in said aqueous phase with an immobilized lectin; and
(b) eluting bound glycoprotein off said immobilized lectin, wherein said lectin is optionally specific for the disease.

37) The method of any of claims 30-35 further comprising identifying secretion sequences in isolated glycoproteins from the aqueous phase.

38) The method of any of claims 30-35 further comprising distinguishing a subset of glycoproteins that are differentially expressed or glycosylated in cells harboring a disease from the glycoproteins that are separated.

39) The method of claims 38 wherein the differentially expressed or glycosylated glycoproteins are distinguished by:
(a) preparing a first glycoprotein expression pattern from the separated glycoproteins; and
(b) subtracting from the first glycoprotein expression pattern a second glycoprotein expression pattern, wherein said second glycoprotein expression pattern is from cells not harboring the disease.

40) The method of claim 39 wherein said second protein expression pattern is prepared by:
(a) providing a plurality of cells harboring a disease;
(b) solubilizing said cells in a liquid medium comprising water and a detergent with a cloud point;
(c) separating said liquid medium into an aqueous phase comprising hydrophilic proteins and a detergent phase comprising lipophilic proteins;
(d) separating glycoproteins from non-glycoproteins in said aqueous phase; and
(e) identifying said separated glycoproteins.

41) The method of claim 38 wherein said differentially expressed separated glycoproteins are identified by proteolysis and liquid chromatography/mass spectrometry (LC/MS).

42) A method of identifying the activity of glycosyltransferases in cells comprising:
(a) providing a plurality of cells;
(b) expressing a recombinant protein comprising, a GPI-anchoring sequence and optionally comprising an affinity tag in said cells;
(c) incubating said cells with an enzyme that cleaves phospholipid anchors to yield cleaved recombinant protein comprising an inositol moiety and optionally an affinity tag, uncleaved recombinant protein, and total cleaved protein;
(d) separating said cleaved recombinant protein from uncleaved recombinant protein and total cleaved protein; and
(e) optionally characterizing a glycosylation pattern on said cleaved recombinant protein.

43) A method to distinguish the disease state of a tissue sample comprising:
(a) removing a tissue sample from a patient; and
(b) recognizing the presence of a disease marker in said tissue sample wherein said disease marker is identified by:
(i) providing a plurality of cells harboring a disease and comprising glycoproteins and non-glycoproteins;
(ii) solubilizing said cells in a liquid medium comprising water and a detergent with a cloud point;
(iii) separating said liquid medium into an aqueous phase comprising hydrophilic proteins and a detergent phase comprising lipophilic proteins;
(iv) separating glycoproteins from non-glycoproteins in said detergent phase; and
(v) characterizing a glycosylation pattern on one or more of said separated glycoproteins.

44) A method to target therapeutic compounds to diseased tissue comprising:
(a) linking a therapeutic compound to an agent which recognizes one or more disease markers wherein said disease markers are identified by:
(i) providing a plurality of cells harboring a disease and comprising glycoproteins and non-glycoproteins;
(ii) solubilizing said cells in a liquid medium comprising water and a detergent with a cloud point;
(iii) separating, said liquid medium into an aqueous phase comprising hydrophilic proteins and a detergent phase comprising lipophilic proteins;
(iv) separating glycoproteins from non-glycoproteins in said detergent phase;
(v) characterizing a glycosylation pattern on one or more of said separated glycoproteins; and
(b) administering said linked compounds to a patient.
